(19) United States
(12) Patent Application Publication
(10) Pub. No.: US 2025/0235154 A1
(43) Pub. Date: Jul. 24, 2025

(54) OBSTRUCTIVE SLEEP APNEA PREDICTION AND ANALYTICAL REASONING USING HYPERPARAMETERS FOR ACCURATE MODELING OF RISK

(71) Applicant: Cerner Innovation, Inc., Kansas City, MO (US)

(72) Inventors: Wasimakram Binnal, Bengaluru (IN); Reshma N, Bengaluru (IN); Pradeep Premakumar, Karnataka (IN); Rajvardhan J P, Karnataka (IN)

(73) Assignee: Cerner Innovation, Inc., Kansas City, MO (US)

(21) Appl. No.: 18/776,012

(22) Filed: Jul. 17, 2024

Related U.S. Application Data
(60) Provisional application No. 63/624,734, filed on Jan. 24, 2024.

(51) Int. Cl. A61B 5/00; G16H 10/60; G16H 40/67; G16H 50/30

(52) U.S. Cl. CPC A61B 5/4818; A61B 5/7267; A61B 5/7275; G16H 10/60; G16H 40/67; G16H 50/30

(57) ABSTRACT

The system and methods for predicting sleep apnea in subjects using machine-learning models. The method entails collecting a dataset from a variety of data sources such as electronic records, sleep audio data, or biometric sensor data from wearable devices. A set of features are extracted from the dataset including demographic features, comorbidities features, anthropometric features, or sleep history features. The method generates a set of compound features by performing transformations on the set of features. The extracted set of features and generated set of compound features are collated into a compound feature vector used for training the machine-learning models. The models accurately predict the sleep apnea intensity score which is mapped to a sleep apnea category such as controlled, mild, moderate, or severe apnea. Based on the predicted sleep apnea category treatment regimens as action strategies are recommended to the users.

### CROSS REFERENCE OF RELATED APPLICATIONS

This application claims priority to U.S. Provisional Patent Application No. 63/624,734, filed on Jan. 24, 2024, titled “OBSTRUCTIVE SLEEP APNEA PREDICTION AND ANALYTICAL REASONING USING HYPERPARAMETERS FOR ACCURATE MODELING OF RISK,” which is incorporated by reference in its entirety for all purposes.

### BACKGROUND

Sleep apnea is a serious sleep disorder in which breathing repeatedly stops and starts during sleep. Obstructive Sleep Apnea is a common type of sleep apnea in which throat muscles relax and block the flow of air into the lungs. When obstructive sleep apnea occurs, the concentration of oxygen in the blood decreases, the concentration of carbon dioxide increases, and the sympathetic nerve is activated due to repeated awakenings during sleep, which sometimes worsens into hypertension, cardiovascular disease, and cerebrovascular disease. Traditional methods to diagnose obstructive sleep apnea use features from polysomnography test results. Polysomnography is a diagnostic test that records and analyzes sleep states to diagnose various abnormal conditions that occur during sleep. However, the laboratories that are equipped to conduct polysomnography tests are not only small in numbers but also not easily accessible because of the shortage of numbers worldwide. Consequently, access to these laboratories is limited. Further, such a test entails that the patient spend at least one night at the laboratory. Such tests can also be expensive and not covered by insurance. Hence, polysomnography tests may not be performed for patients suspected of having obstructive sleep apnea due to procedural, logistic, inconvenience, financial, or other reasons.

Assistive devices-such as Positive Airway Pressure (PAP), mechanical ventilators, or Continuous Positive Airway Pressure (CPAP) devices-greatly help in maintaining normal respiratory functions. However, these devices may also cause harm and distress to a subject because of stress or strain due to the amount of pressure exerted on the respiratory system of a subject. These devices are reactive and hence are not capable of proactively predicting and identifying respiratory distress or discomfort. Thus, it is important to identify patients who would benefit from these techniques using an approach with high-precision metrics.

Further, early detection and treatment of sleep apnea can also help in lowering the risk of chronic heart disease, mortality, cancer, depression, Alzheimer's disease, Endocarditis, high blood pressure, and diabetes.

### SUMMARY

In some embodiments, a computer-implemented method is provided to determine sleep apnea prediction for a subject using machine learning models. The method includes collecting an input dataset from one or more data sources for a subject and extracting a set of features from the dataset. The method generates a set of compound features by using the set of features extracted from the dataset by performing arithmetic operations on two or more features. The set of features and compound features are collated into a compound feature vector for training a model. The method uses a trained machine learning model to predict intensity score of sleep apnea for a subject by processing the compound feature vector and generates an action strategy based on the predicted intensity score of the sleep apnea. The method produces an output result that represents the action strategy and corresponding action strategies can be recommended to a user.

In an aspect of the present disclosure, one or more data sources can be electronic health records, sleep audio or biometric sensors data from wearable devices. The method includes extracting a set of features from the input dataset from one or more data sources. When the input data source is electronic health records, extracted set of features may include demographic features (e.g. age, gender), comorbidities features (does the subject have diabetes and hyper-tension?), anthropometric features (e.g. BMI), or sleep history features (average sleep hours). The sleep audio data may include sound analysis, snoring events etc. while biometric sensors data can include electrocardiogramata, heart rate, blood pressure, body and skin temperature, respiration rate etc. captured by the wearable device. After the set of features are extracted, a compound feature is generated from two or more features (e.g., by performing arithmetic or statistical operation on two or more features). A compound feature in a set of compound features may include (for example) chem_age_med which is calculated by measuring amount of O2 level (blood oxygen saturation) across different types of blood test and multiplying by the subject age groups across median O2 level (median oxygen level), where amount of O2 level across different types of blood test and subject age groups are the features extracted from the electronic health records.

The method may further include preprocessing the input dataset during which normalized data may be generated using various normalization techniques, such as z-score normalization, linear normalization, or standard deviation normalization etc. Data preprocessing may also prepare, clean, and/or maintain the quality of the data and may involve tasks such as handling one or more missing values, converting a categorical set of features to a numerical set of features, correcting data anomalies and/or removing noise. After data preprocessing, the extracted set of features and generated set of compound features are collated into a compound feature vector as training dataset which is used to train a machine-learning model and/or underlying algorithms.

The method may further include predicting, for the subject, an intensity score for sleep apnea by processing the compound feature vector using a machine learning model. The machine learning model is trained on the compound feature vector associated with each subject as input variables. The machine learning model may learn which features strongly correlate with the prediction of sleep apnea and affect it. Any supervised machine learning model can be deployed such as Random Forest, Support Vector Machine, AdaBoost, and Regression models. By studying the various patterns of compound feature vectors, the model generates predictions to determine the intensity of the sleep apnea. The intensity score of sleep apnea, generated by the machine learning model, may be mapped using thresholds to a category of sleep apnea. A set of categories of sleep apnea may include controlled apnea, mild apnea, moderate apnea, and severe apnea signifying the severity of sleep apnea in a subject. Machine learning models may also generate classification categories of sleep apnea as an output. Based on the intensity score or classification categories predicted by the machine learning model, appropriate action strategies are recommended to a user.

In some embodiments, a system is provided that includes one or more data processors and a non-transitory computer-readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 is an illustrative block diagram of a computer-implemented method that may be utilized for predicting sleep apnea using machine learning models.

FIG. 2 and FIG. 3 are example illustrations of calculating two compound features that are; ‘chem_age_med’, determined by multiplying the concentration of O₂ levels in various blood tests by age group of the subject; and ‘chem_weight_med’, calculated by multiplying the O₂ proportion in hemoglobin by the weight class of a subject.

FIG. 4 illustrates the steps of gathering the data from different data sources followed by data preprocessing, feature extraction, and classification using a machine learning model to predict sleep apnea.

FIG. 5 illustrates the step-by-step process including collecting data of a subject, preprocessing data, extracting prominent features, and training machine-learning models. The trained machine-learning model generates the classification categories of sleep apnea and an action strategy.

FIG. 6 illustrates the process flow of using compound feature vectors to train machine-learning models. The model generates a sleep apnea intensity score, which is then mapped to sleep apnea classification categories and generates respective action strategies.

FIG. 7 illustrates another example process flow of the present disclosure, where a decision tree classifier can be used to generate sleep apnea classification categories using the compound features vector and generate respective action strategies.

FIG. 8 shows a dashboard for displaying insights into the subject's condition, by displaying the sleep apnea intensity score, classification category, and appropriate action strategies recommended for the respective severity level of sleep apnea.

FIG. 9 illustrates an example flow of a method for obtaining the final output result of an action strategy using a machine-learning model trained on a set of compound features in accordance with some embodiments of the present disclosure.

FIG. 10 illustrates a simplified diagram of a distributed system for implementing the method of FIG. 1.

FIG. 11 illustrates a simplified block diagram of a cloud-based system environment in which various service of servers may be offered as cloud services.

FIG. 12 illustrates an example architecture of a computing system that can implement at least one example of the disclosed method.

FIG. 13 illustrates an exemplary server which may include a processor, a memory, and a mass storage device.

In the appended figures, similar components and/or features may have the same reference label. Further, various components of the same type may be distinguished by following the reference label with a dash and a second label that distinguishes among the similar components. If only the first reference label is used in the specification, the description applies to any one of the similar components having the same first reference label irrespective of the second reference label.

### DETAILED DESCRIPTION

The ensuing description provides preferred exemplary embodiment(s) only and is not intended to limit the scope, applicability, or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as outlined in the appended claims.

In some embodiments of the present disclosure, techniques are provided to use machine-learning models to predict obstructive sleep apnea without using the results of a polysomnography diagnostic test. Extract, Transform, and Load (ETL) processes may be used for collecting a dataset from different sources for a subject, transforming it into a standardized format, and loading it into a centralized storage medium or data repository. Data sources include data from electronic health records, audio data (e.g., sound analysis, snoring, etc.) and in addition may also include biometric data from a sensor in a wearable device (e.g., electrocardiogra (ECG) data, heart rate, blood pressure, body and skin temperature, respiration rate, etc.).

For example, if the input dataset is an electronic health record, a set of features that are extracted may include demographic features, comorbidities features, anthropometric features, or sleep history features. A feature in the set of features may include (for example) BMI (body mass index), sleep_walking (does a subject sleepwalk?), bp_sleep (approximate number of times individuals recall waking up in daily sleep and diagnosed with hypertension), diabetic_sleep (approximate number of times individuals recall waking up in daily sleep and is diagnosed with diabetes), etc. When the input data source includes biometric data from a sensor in a wearable device, features such as electrocardiogramata, heart rate, blood pressure, body and skin temperature, respiration rate, etc. may be extracted and processed to train the machine-learning models to detect obstructive sleep apnea.

In some embodiments, analog data in the form of audio inputs from suitable recording devices (e.g., specialized microphones placed around the subject or using smart mobile devices) may also be sampled, converted to the digital format by considering factors, e.g., sampling rate and bit depth, and subsequently stored in a memory on the device or in the cloud. The audio samples can be preprocessed, for example, to enhance audio quality and to remove noise from audio data. Subsequently, features may be extracted from the audio data. The features may be defined to represent the breathing patterns of a subject during sleep (e.g., that otherwise are to be estimated by explicitly asking questions to subjects). Subsequently, audio features can be extracted by preprocessing raw audio (e.g., using MFCCs, spectrograms, or machine-learning based features) to transform audio data into feature vectors. Features from the snoring audio data can help in better understanding the breathing patterns of a subject, or the quality of sleep, that otherwise are coarsely estimated by asking questions explicitly to subjects.

The method generates a set of compound features from two or more of the set of features by performing arithmetic operations on two or more of the set of features. A compound feature in the set of compound features may include (for example) chem_age_med which is calculated by measuring amount of O2 level (blood oxygen saturation) across different types of blood tests and multiplying by the subject age groups across median O2 levels (median oxygen levels); hem_weight_med is calculated by measuring O2 level in the hemoglobin of a subject and multiplying it by weight class of the subject across median O2 level where weight class is obtained by discretizing weight into 4 classes; and oxy_age_bmi is calculated by multiplying average O2 levels of a subject with the age of the subject's group and dividing by BMI etc. The extracted set of features and generated set of compound features are collated into a compound feature vector which may be used to train a machine-learning model and/or underlying algorithms.

The embodiments of the present disclosure may be utilized for feature generation using the compound feature vector. Feature generation is used to increase the number of variables by using various statistical tests and is based on a deep feature synthesis algorithm. The machine-learning model is trained on the compound feature vector. Any supervised machine-learning model (e.g., Random Forest model, Support Vector machine model, AdaBoost model) can be used. By studying the various patterns of compound feature vectors, the model generates predictions to determine the intensity of the sleep apnea experienced by the subject. Based on the intensity score or classification category, predicted by the machine-learning model, appropriate remedial action strategies are recommended to the user. The users may include healthcare providers and a set of subjects. Non-limiting examples of action strategy include making preventive recommendations for therapies (e.g., recommending healthy lifestyle changes, getting regular physical activity, maintaining healthy sleeping habits and a healthy weight, limiting alcohol and caffeine intake, and quitting smoking) for the subjects predicted with controlled or mild apnea. Medications (e.g., Acetazolamide), use of oral or breathing devices, etc. for the subjects predicted with moderate or severe apnea. Based on the model predictions, the severity of sleep apnea is determined, and an action strategy is recommended so that healthcare providers can make informed decisions about the condition of the subject. By maintaining a feedback loop, subjects are regularly updated on their sleep apnea condition and the success of different interventions recommended by the provider. It may also inculcate a sense of involvement in the management of sleep apnea when subjects feel empowered to make educated decisions about their health and well-being and to take precautionary measures before time.

### Overview

Sleep apnea is a common condition in which breathing stops and restarts many times during sleep. This can prevent the body from getting enough oxygen. Obstructive sleep apnea can be one of the most common types of sleep apnea and happens when the upper airway becomes blocked many times during sleep, reducing or completely stopping airflow. When obstructive sleep apnea occurs, the concentration of oxygen in the blood decreases, the concentration of carbon dioxide increases, and the sympathetic nerve is activated due to repeated awakenings during sleep, which sometimes worsens into hypertension, cardiovascular disease, and cerebrovascular disease. Traditional methods to diagnose obstructive sleep apnea use polysomnography results, which is a diagnostic test that records and analyzes sleep states to diagnose various abnormal conditions that occur during sleep. However, the laboratories equipped to conduct polysomnography tests are limited and not easily accessible. Consequently, access to these laboratories is restricted. Further, such a test entails that the patient spend at least one night at the laboratory. Hence, polysomnography tests may not be performed for various patients who may be suspected of having obstructive sleep apnea due to procedural, logistic, inconvenience, financial, or other reasons.

In some embodiments, techniques are provided to use machine-learning models to predict obstructive sleep apnea without using the results of a polysomnography diagnostic test. A set of features are extracted from electronic records which may include demographic features, comorbidities features, anthropometric features, or sleep history features. The method generates a set of compound features from two or more features by performing arithmetic operations on the subset of features. For example, hem_weight_med compound feature is computed by multiplying an O₂ proportion in the hemoglobin feature and a weight class feature of the subject.

The method employs data preprocessing techniques and feature extraction techniques for cleaning the dataset and selecting relevant features, respectively. The method uses a machine-learning model trained on the set of compound features as input variables. It generates the intensity score or classification category of the sleep apnea as an output. The intensity score generated by the machine-learning model is then mapped to a category of one or more categories of sleep apnea including controlled apnea, mild apnea, moderate apnea, or severe apnea. Based on the identified category, the method generates an action strategy that includes recommending one or more preventive strategies for the subject who is predictively diagnosed with mild apnea; or recommending oral or breathing devices strategies for the subjects predicted with moderate or severe apnea.

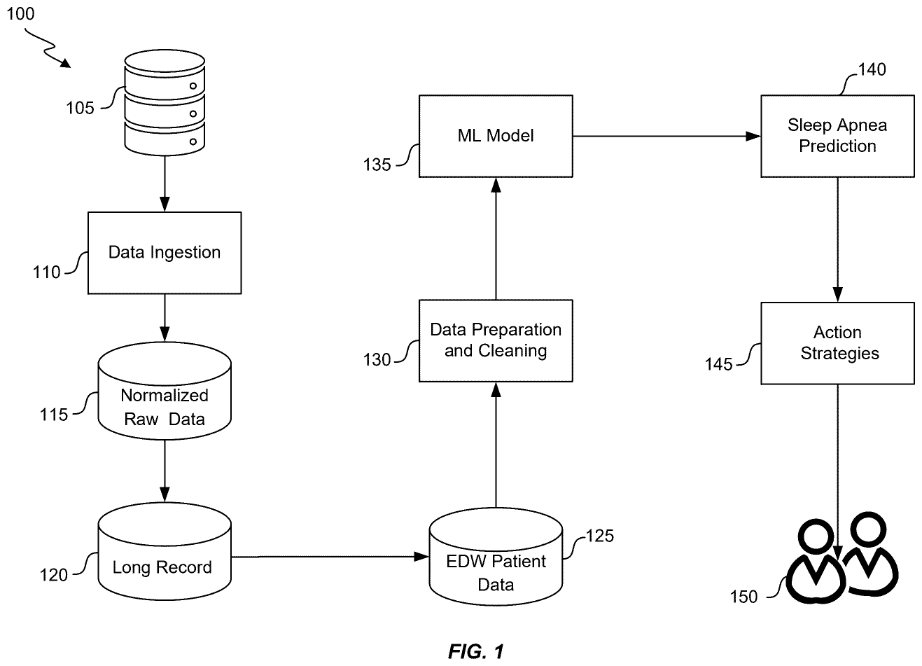

FIG. 1 is an example illustrative block diagram of a computer-implemented method that can be utilized for predicting obstructive sleep apnea using trained machine-learning models. The example embodiment may be implemented by a system 100 that collects an input dataset from a variety of data sources and stores the dataset in a data repository and a database 105, a data ingestion 110, a normalized data 115, a long record 120, an EDW patient data 125, a data preparation and cleaning 130, an ML model 135, a sleep apnea prediction 140, the action strategies 145, and a user 150.

A data repository and a database 105 present within a computing system or a data repository can be used to store the dataset. The dataset can be collected from a variety of data sources. The data sources include data from Electronic Medical Records (EMR), electronic health records, and Electronic Health Records (EHR), or may also include biometric data from wearable sensor devices. The data may include text data, time-series data, and audio data. If the input data source is electronic health records, a set of features is extracted which may include demographic features, comorbidities features, anthropometric features, and sleep history features. These sets of features can be in the form of text format or can be in the form of time series data captured over different time intervals. A feature in the set of features may include (for example) Body Mass Index (BMI), sleep_walking (does a subject sleepwalk?), bp_sleep (approximate number of times individuals recall waking up in daily sleep and are diagnosed with hypertension), diabetic_sleep (approximate number of times individuals recall waking up in daily sleep and are diagnosed with diabetes), etc.

Data ingestion 110 may be used to facilitate the orderly transfer of data from the data source to the subsequent stages of the data wrangling process. This can help in maintaining the integrity and consistency of data. Data ingestion combines data from different data sources, using unique identifiers to avoid discrepancies, and utilizing gender and age as demographic identifiers, thus enabling the process to exploit diverse information.

Normalized data 115 receives the collected dataset from data ingestion 110 process. It involves the transformation of data into a consistent normalized format, eliminating variations in the data representation, which makes the data suitable for analysis and comparison. The normalized data can be generated using various normalization techniques and algorithms that may include z-score normalization which normalizes each data point using mean and standard deviation, linear normalization which normalizes each data point using minimum and maximum feature values present in the data, and standard deviation normalization which uses standard deviation to normalize each data point, etc. This step transforms the raw data in various formats into standardized data. After normalization, the data may be passed through various series of data wrangling blocks that involve data parsing, conversion, or transformation to extract the relevant data.

Long records 120 receives normalized data 115. Long records 120 can encompass extended time frames or summarized patient information, making it feasible to pursue the subsequent steps.

EDW patient data 125 (Enterprise Data Warehouse) stores the transformed dataset of the subjects. A data warehouse is a centralized repository where organized data, historical or newly generated, is stored, examined, and retrieved. To provide a more comprehensive picture of the medical history and conditions of the subject, this step may entail integrating the processed data with EDW patient data 125. It may include Extract, Transform, and Load (ETL) processes for collecting subject data from different sources, transforming it into a standardized format, and loading it into a centralized enterprise data warehouse. The EDW patient data ensures data quality and consistency, tracks historical data, provides a unified view of data from various sources, simplifies data integration, and facilitates prediction analysis.

Data preparation and cleaning 130 can prepare, clean, and maintain the quality of the data received from EDW patient data 125. The module involves tasks like handling one or more missing values, converting a categorical set of features to a numerical set of features, correcting data anomalies, and removing noise. The missing values can be handled by using techniques such as imputation and data removal. The conversion of the categorical set of features to the numerical set of features can be achieved using various techniques, and algorithms that may include One-Hot Encoding, Label Encoding, Binary Encoding, Helmert Encoding, etc. Statistical methods such as mean, median, and quantiles can be used to detect anomalies and noise in the dataset. In addition, data visualization and exploratory data analysis techniques can also be used to detect anomalies and remove noise in the dataset. Clean data can then be used to train machine-learning models. The data preparation can involve generating a set of compound features from two or more of the set of features. The extracted set of features and generated set of compound features are collated into a compound feature vector which may be used to train a machine-learning model and/or underlying algorithms.

ML model 135 receives the compound feature vector data from data preparation and cleaning 130 and includes a machine-learning model that enables the prediction of sleep apnea. The machine-learning model is trained on the set of compound features associated with each subject as input variables. In an embodiment, the machine-learning model is based on learned correlations between a set of features extracted from different data sources and a set of compound features generated using the set of features. The machine-learning algorithm may learn which features strongly correlate and affect the prediction of sleep apnea. The model can generate a sleep apnea intensity score as an output or can generate classification categories of sleep apnea. Any supervised machine-learning model can be deployed. Examples of supervised machine-learning models that can be used include the Random Forest model, Support Vector machine model, AdaBoost model, KNN, Regression, etc. By studying the various patterns of compound feature vectors, the model generates predictions to determine the intensity of the sleep apnea.

Sleep apnea prediction 140 generates a prediction of a category of sleep apnea based on the output generated by ML model 135. This can involve using thresholds to map the intensity score generated by the machine-learning model to a category of sleep apnea. A set of categories of sleep apnea includes controlled apnea, mild apnea, moderate apnea, and severe apnea signifying the severity of sleep apnea in a subject. The controlled apnea means that the apnea and hypopnea rate of the subject referred to as the apnea/hypopnea index (AHI) is within the normal range. Mild apnea is the least-advanced form of obstructive sleep apnea and means a person has an AHI between 5 and 15 i.e., a subject has between 5 and 15 apnea or hypopnea events per hour. In the case of the moderate apnea category of sleep apnea, a subject has 15 to 30 apnea or hypopnea events per hour; while in the case of the severe apnea category of sleep apnea, a subject has more than 30 apnea or hypopnea events per hour, and this is the most severe form of sleep apnea among the set of categories of sleep apnea described above.

Action Strategies 145 generates an appropriate action strategy for the subject based on the severity of the sleep apnea predicted by the model. Non-limiting examples of action strategy include making preventive recommendations for therapies (e.g., recommending healthy lifestyle changes, doing regular physical exercises and activities, maintaining healthy sleeping habits and a healthy weight, limiting alcohol and caffeine intake, or quitting smoking) for the subject's predicted with controlled or mild apnea; recommending medications (e.g., Acetazolamide) and use of oral or breathing devices etc. for the subjects who are predicted with moderate or severe apnea or other treatment programs for subjects who are at a higher risk of developing sleep apnea. The model's predictions and related parameters such as intensity score are used to determine the appropriate approach according to the intensity of sleep apnea diagnosed in the subject.

Users 150 receive the action strategies. The users can include healthcare providers, subjects, or disease surveillance public health departments that have an intrinsic interest in getting the risk surveillance data of chronic diseases. Based on the model predictions, severity of sleep apnea, and action strategy recommended, the healthcare providers can make informed decisions about the condition and the remedial treatment pathways of the subject. This can lead to creating an effective and efficient plan to provide care to each subject including adherence to prescribed treatment programs outlined in the gold standard guidelines. The model predictions, severity of sleep apnea, and recommended action strategies can also be integrated into the subject's healthcare electronic records or made available via customized dashboards.

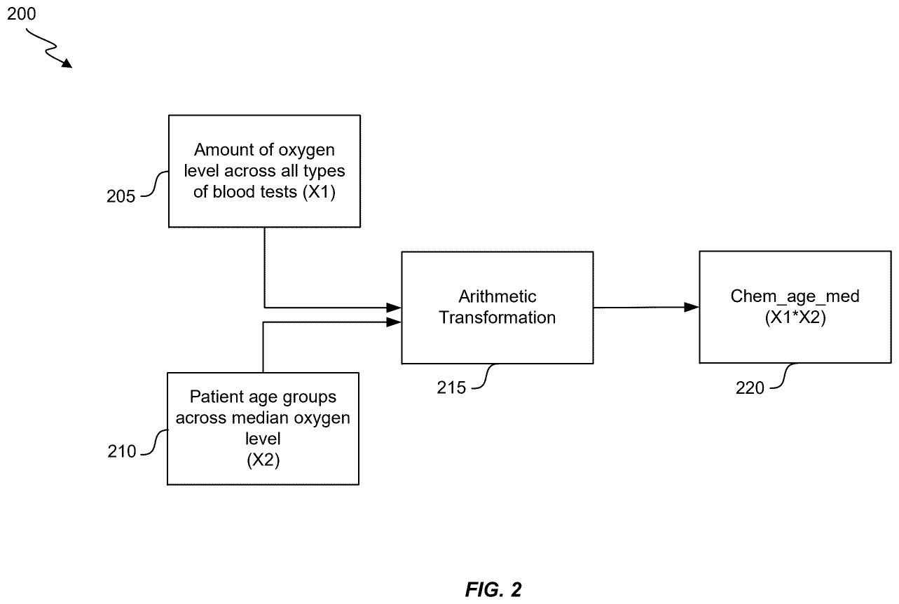

FIG. 2 is an example illustration of the generation of a compound feature 200 using two or more features. For example, an example feature measuring the amount of oxygen level across different types of blood tests 205 (i.e. oxygen blood saturation), herein referred to as X1, and another feature patient age group across median oxygen level 210, herein referred to as X2, are passed to arithmetic transformation 215 to generate a compound feature Chem_age_med 220, herein referred to as X1*X2 by multiplying the feature value of amount of oxygen level across different types of blood tests and feature value of patient age group across median oxygen level. Arithmetic transformation 215 can perform a set of operations on the set of features including but not limited to arithmetic operations such as addition, subtraction, multiplication, division, and statistical operations including mean, median, mode, percentiles, etc. As another example, med_sleep_age compound feature is generated by measuring the approximate median of the number of times sleep was without fatigue multiplied by three age groups. As another example, percentile_high_bmi compound feature is generated by calculating the 90th percentile score for different patients suffering from sleep apnea for each subject and multiplying it with the BMI feature of the subject.

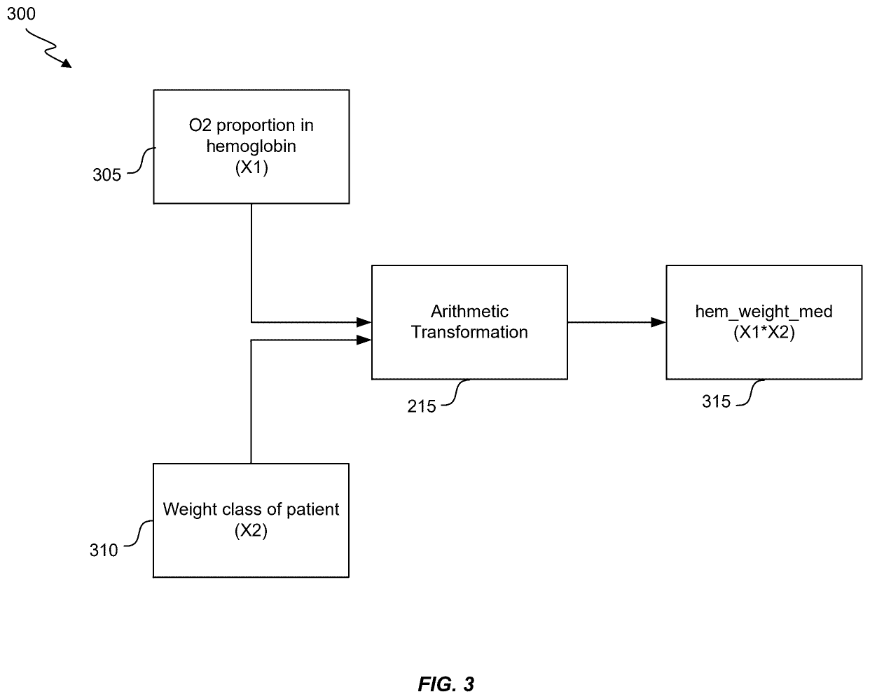

FIG. 3 is another example illustration of the generation of another compound feature 300 using two or more features. For example, features such as O₂ proportion in hemoglobin 305, herein referred to as X1, and another feature weight class of a patient 310 (discretized into four classes), herein referred to as X2, are passed to arithmetic transformation 215 to generate a compound feature hem_weight_med 315, herein referred to as X1*X2 by multiplying the feature value of O₂ proportion in hemoglobin and feature value of weight class of a patient (discretized into four classes). As another example, compound features such as med_comorbities_oxy is calculated by dividing the weight-based comorbidities feature by the median value of the O₂ levels feature. The oxygen levels in the blood can typically be measured by using a pulse oximeter or can be assessed in a laboratory context to determine chemical proportions, such as those in hemoglobin and nitrogen. These features can offer insights into the deviation of each subject's features from that of the averages of subjects in the dataset, utilizing O₂ levels as a metric. Taking advantage of the transforming ability of machine-learning, our model can integrate these features, alongside a wide array of generic subject biomedical data, to calculate the intensity score which can help in estimating the intensity of sleep apnea and categorizing the subject according to its category of sleep apnea.

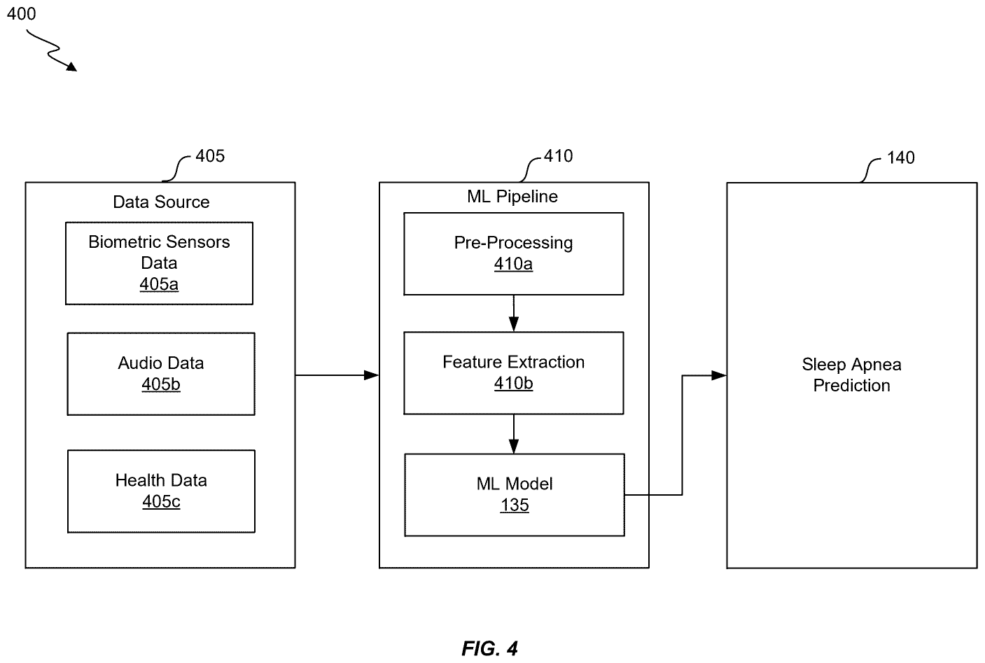

FIG. 4 illustrates the steps of preparing the dataset from different data sources followed by a process of data preprocessing, feature extraction, and classification using an ML model to predict sleep apnea. The example embodiment may be implemented by system 400 which includes a data source 405, an ML pipeline 410, and a sleep apnea prediction 140. Data source 405 may further include biometric sensors data 405a, audio data 405b, and health data 405c. ML pipeline 410 may further include a preprocessing 410a, a feature extraction 410b and machine-learning classification 410c.

Data source 405 collects a dataset from different data sources for a subject and extracts the relevant features from the data source. Biometric sensors data 405a includes data from a sensor in a wearable device (e.g., electrocardiogramata, heart rate, blood pressure, body and skin temperature, respiration rate etc.). If a subject is wearing sensors such as pulse oximeter, additional features like the oxygen levels in the blood can be measured as well.

Audio data 405b includes audio data (e.g., sound analysis, snoring, etc.). Analog data in the form of audio inputs from suitable recording devices, such as specialized microphones placed around the subject or using smart mobile devices, may also be sampled, and converted to the digital format by considering factors e.g., sampling rate and bit depth, and subsequently stored in a memory on the device or in the cloud. The audio samples can be preprocessed, for example, for enhancing audio quality and to remove noise from audio data, different noise reduction techniques can be utilized. Subsequently, relevant features may be extracted from the audio data to capture the breathing patterns of a subject during sleep that otherwise are to be estimated by explicitly asking questions to subjects. Subsequently, audio features can be extracted by preprocessing raw audio (e.g., using MFCCs, spectrograms, or machine-learning based features) to transform audio data into feature vectors. Audio features defining the acoustic characteristics of apnea or hypopnea are extracted from the sleep respiration sounds. Features related to snoring that are extracted from raw audio can include inter-event silence (count of number of long silences between snore events), running variance (measures the inter-snore variability of the score energy over the night), apneic phase ratio (measures the relative duration of the upper airway collapse), pitch density (measuring the stability of the tissue's vibration frequency) and Mel-cepstral stability (measures the stability of the spectrum of the night). Using the extracted features from the audio data, deep learning models can be employed to detect snore events and non-snore events. In addition to snoring features, additional features can be extracted from raw audio including the approximate average number of times the subject wakes up in the night, the approximate average duration of deep sleep, etc.

Health data 405c data includes data from EHR and EMR. A set of features is extracted from electronic records which include demographic features, comorbidities features, anthropometric features, or sleep history features. Demographic features include age, race, gender, marital status, ethnicity, etc. Comorbidity describes the existence of more than one disease or condition in a subject at the same time. Comorbidity features extracted for a subject can include chronic diseases such as diabetes and hypertension, the number of comorbidities in a subject, etc. Anthropometric features are noninvasive quantitative measurements of the subject's body that can include height, weight, head circumference, BMI, body circumferences to assess for adiposity (waist, hip, and limbs), skinfold thickness, etc. Sleep history features can include the average number of sleep hours of a subject, the approximate number of times individuals recall waking up in daily sleep, etc. These various forms of inputs demonstrate the capability of the present disclosure to work with the dataset including multimodal data formats for example, text, audio, or a combination to provide a rich dataset that is used for generating compound features and training the machine-learning models using the training dataset.

After the collection of a set of features for a subject, it can be processed, and the compound features are generated. The set of features and the compound features may be stored in a database i.e., Oracle Cerner Database. These stored features may be used later to generate predictions and devise strategies for healthcare professionals. This may enable these professionals to make well-informed decisions, providing high-quality and value-based patient care by refining the accuracy and effectiveness of the model. This approach does not merely streamline the diagnosis and monitoring process but also holds the potential to facilitate early interventions and can enhance the overall quality of life for patients who may be at risk of suffering from sleep apnea.

ML pipeline 410 preprocesses the data received from data sources 405, extracts the relevant features, and generates sleep apnea prediction using the machine-learning models. Data preprocessing 410a can include tasks like data cleaning, data integration, and data transformation to improve the quality of the data. The data cleaning tasks can include identifying and correcting errors or inconsistencies in the data such as missing values, outliers, and duplicates. Various techniques can be used for data cleaning such as imputation, removal, and transformation. Statistical techniques such as mean, median, and quantiles can be used to detect anomalies and noise in the dataset. In addition, data visualization and exploratory data analysis techniques can also be used to detect anomalies and remove noise in the dataset. Clean data can then be used to train the machine-learning model. The data integration involves combining data from multiple data sources to create a unified dataset. Data integration can be challenging as it entails handling data with different formats, structures, and semantics. Techniques such as record linkage and data fusion can be used for data integration. The data transformation involves converting the data into a suitable format for analysis. Multiple techniques can be used such as normalization, standardization, encoding, and discretization. Normalization is used to scale the data to a common range and standardization is used to transform the data to have zero mean and unit variance. Discretization is used to convert continuous data into discrete categories. Encoding is used to convert a categorical set of features to a numerical set of features and can be achieved using various techniques and/or algorithms that may include One-Hot Encoding, Label Encoding, Binary Encoding, Helmert Encoding, etc.

Feature extraction 410b extracts the relevant features from the preprocessed dataset passed from preprocessing 410a. This may include extracting a set of features such as demographic features, comorbidities features, anthropometric features, and sleep history features from electronic medical records. A feature in the set of features may include (for example) BMI, sleep_walking (does a subject sleepwalk?), bp_sleep (approximate number of times individuals recall waking up in daily sleep and diagnosed with hypertension), diabetic_sleep (approximate number of times individuals recall waking up in daily sleep and diagnosed with diabetes), etc. Feature extraction 410b can extract relevant biometric features from sensor data in a wearable device such as ECG data, heart rate, blood pressure, body and skin temperature, respiration rate etc. In addition, feature extraction 410b can also extract features related to sleep analysis and snoring from sleep audio data. Feature extraction collates the extracted set of features and generated compound features into a compound feature vector.

Feature extraction 410b may also include tasks such as feature generation which is used to increase the number of variables by using various statistical tests and is based on a deep feature synthesis algorithm. Deep feature synthesis is an algorithm that creates features between sets of relational data to automate the machine-learning process. The algorithm applies mathematical functions to multiple datasets to transform them into new groups with better features. Feature extraction 410b evaluates the importance of the set of features and generated compound features and calculates feature importance as weights for each feature. The features can have different weights in predicting the output, where the sum of the total weightage of the features will be equal to 1. The relevant features are the ones that have high weightage while calculating the feature importance. The relevant set of features and compound features are collated in the compound feature vector.

ML model 135 uses the compound feature vector passed from 410b to train a machine-learning model. The model uses historical data to identify patterns and relationships present in the data. The machine-learning model is trained on the set of compound features associated with each subject as input variables and is based on learned correlations between features extracted from different data sources and compound features generated using the set of features. The machine-learning algorithm may learn which features strongly correlate and affect the prediction of sleep apnea. Any supervised machine-learning model can be deployed. Examples of supervised machine-learning models that can be used include Random Forest model, Support Vector machine model, AdaBoost model, KNN, Regression, etc. For example, machine-learning models such as Linear Regression can be used to predict the intensity of sleep apnea using the compound features vector as input variables. The regression model uses compound features as independent variables of the model and intensity score as a dependent variable during the training. The regression model then learns the coefficients of each compound feature and measures the strength of the relationship between each compound feature and the intensity score. By studying the various patterns of the compound feature vectors, the model generates an intensity score to measure the severity of sleep apnea in the subjects.

Sleep apnea prediction 140 generates a prediction of a category of sleep apnea based on the output generated by ML model 135. This can involve using thresholds to map the intensity score generated by the machine-learning model to a category of sleep apnea. A set of categories of sleep apnea includes controlled apnea, mild apnea, moderate apnea, and severe apnea signifying the severity of sleep apnea in a subject.

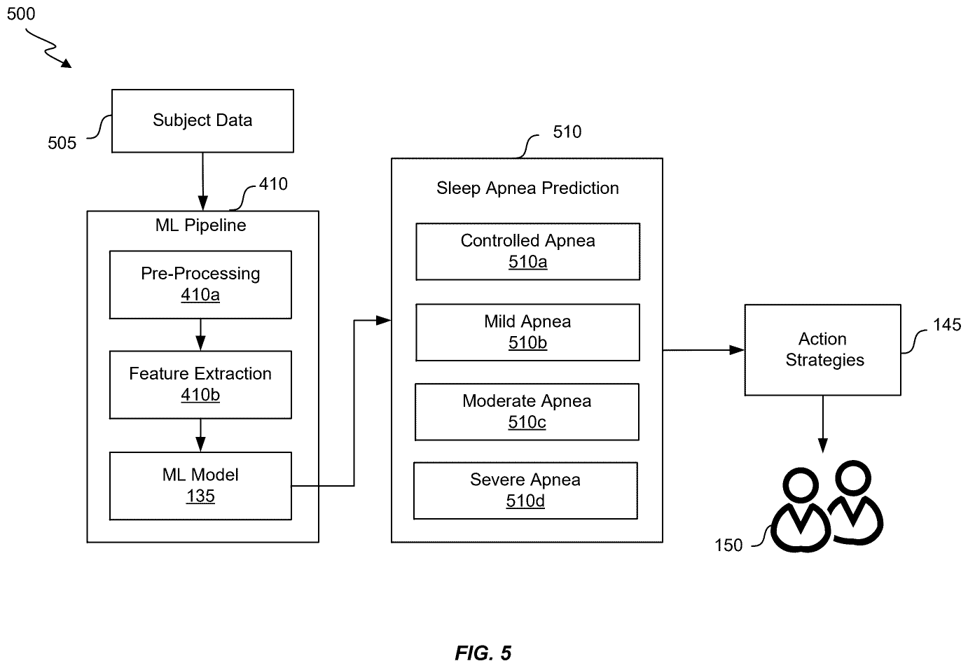

FIG. 5 illustrates the step-by-step process including collecting the subject's data, preprocessing, extracting prominent features, and training machine-learning models. The trained machine-learning model generates the classification labels of sleep apnea and an action strategy. The example embodiment may be implemented by system 500 that includes a subject data 505, ML pipeline 410, a sleep apnea prediction 510, action strategies 145 and users 150. ML pipeline 410 may further include preprocessing 410a, feature extraction 410b, and ML model 135. Sleep apnea prediction 510 may further include a controlled apnea 510a, a mild apnea 510b, a moderate apnea 510c, and a severe apnea 510d.

Subject Data 505 collects the subject's medical data from a variety of data sources including EMR, EHR, sleep audio data, and may also include biometric data from sensors of wearable devices worn by the subjects. The data is then passed on to ML pipeline 410 which includes tasks like data preprocessing, feature extraction, and using ML models to generate sleep apnea predictions.

Sleep apnea prediction 510 generates a prediction of a category of sleep apnea based on the output generated by ML model 135. The categories signify the severity of sleep apnea in the subject. The example embodiment may be implemented by sleep apnea prediction 510 that includes predicted categories of sleep apnea such as controlled apnea 510a, mild apnea 510b, moderate apnea 510c, and severe apnea 510d. Controlled apnea 510a means that the apnea and hypopnea rate of the subject (apnea/hypopnea index AHI) is within the normal range. Controlled apnea 510a includes the cases where the subject has no apnea and the cases where the apnea and hypopnea event rate in the subject is less than 5. Mild apnea 510b is the least-advanced form of obstructive sleep apnea and means a person has an AHI between 5 and 15 i.e., he has between 5 and 15 apnea or hypopnea events per hour. In the case of moderate apnea 510c category of sleep apnea, a subject has 15 to 30 apnea or hypopnea events per hour; while in the case of severe apnea 510d category of sleep apnea, a subject has more than 30 apnea or hypopnea events per hour and is the most severe form of sleep apnea among the set of categories of sleep apnea described above.

Action Strategies 145 generates an appropriate action strategy for the subject based on the severity and category of sleep apnea predicted by Sleep Apnea Prediction 510. Users 150 receive the action strategies. The users can include healthcare providers and the set of subjects. Based on the model predictions, severity of sleep apnea, and action strategy recommended, the healthcare providers can make informed decisions about the condition of the subject.

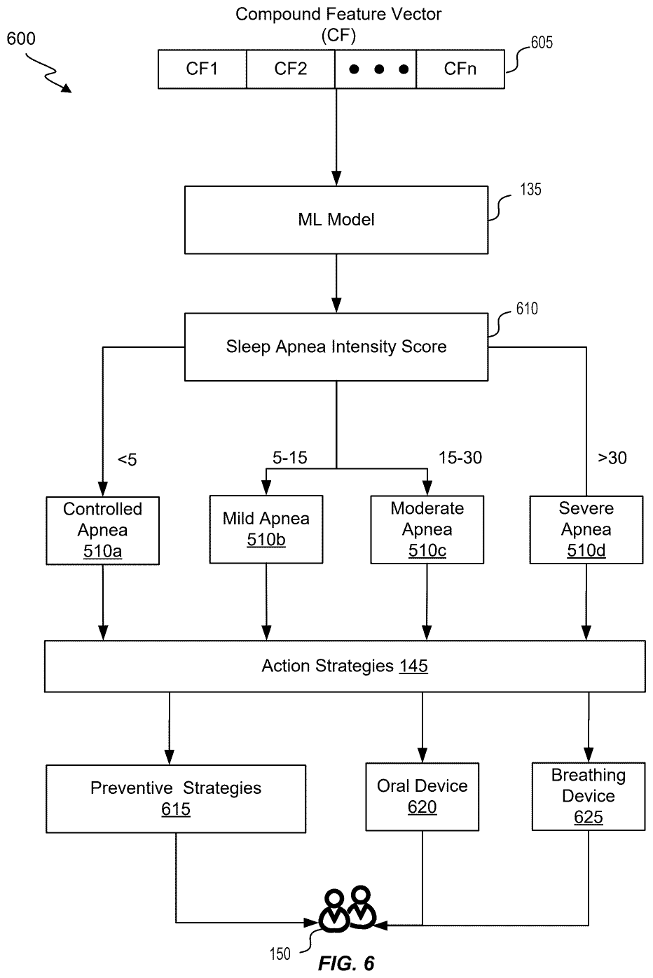

FIG. 6 illustrates the process flow of using a compound feature vector to train the machine-learning model. The model generates a sleep apnea intensity score which is then mapped to a respective sleep apnea classification category and generates a respective action strategy. The example embodiment may be implemented by system 600 includes a Compound Feature vector (CF) 605, ML model 135, a sleep apnea intensity score 610, sleep apnea categories including controlled apnea 510a, mild apnea 510b, moderate apnea 510c, or severe apnea 510d. Action Strategies 145 may comprise a set of preventive strategies 615 or recommending the use of an oral strategy 620 or a breathing device 625 to users 150.

Compound Feature vector (CF) 605 combines the set of features and compound features into a vector. The set of features is extracted from different data sources including electronic medical records, sleep audio data, and biometric sensor data from wearable devices. The set of features is preprocessed, and the relevant set of features is extracted. One or more compound features are generated by performing arithmetic operations on two or more features. Relevant sets of features and generated compound features are combined in a compound feature vector. Compound feature vectors can include features such as oldAge_eye_fatigue (measuring a level of eye fatigue ranging from 0 to 10, measured for higher age group of 50+), diabetic_sleep (approximate number of times individuals recall waking up in daily sleep and diagnosed with diabetes), age_bmi (individuals BMI value is multiplied by age groups), med_comorbities_oxy (weight-based comorbidities divided by median O2 levels) etc. The compound feature vector is then passed to ML model 135. ML model 135 uses compound feature vectors as training dataset to train the model and may learn which features strongly correlate and impact the sleep apnea score. The model predicts a sleep apnea intensity score 610 for each subject of the set of subjects.

Sleep apnea intensity score 610 measures the severity of sleep apnea of the subject based on the output generated by ML model 135. Sleep apnea intensity score measures the rate of the events of apnea and hypopnea per hour of the subject during sleep. If the apnea and hypopnea rate of the subject (apnea/hypopnea index AHI) is within the normal range i.e., the value is <5 then the intensity score is mapped to controlled apnea 510a category. If apnea and hypopnea rate of the subject (apnea/hypopnea index AHI) is between 5 and 15, then the intensity score is mapped to mild apnea 510b category. If apnea and hypopnea rate of the subject (apnea/hypopnea index AHI) is between 15 and 30, then the intensity score is mapped to moderate apnea 510c category. If apnea and hypopnea rate of the subject (apnea/hypopnea index AHI) is greater than 30, then the intensity score is mapped to severe apnea 510d category. Action strategies 145 are generated based on sleep apnea intensity score 610 are generated by the machine-learning model and are mapped to relevant categories of sleep apnea.

Preventive strategies 615 recommends preventive strategies to users 150, if the sleep apnea predicted by the model for the subject is controlled apnea or mild apnea. Controlled apnea means that the apnea and hypopnea rate of the subject is in the normal range. In the preventive strategies, a health care provider can recommend healthy lifestyle changes that can be effective to treat or avoid sleep apnea. These can include getting regular physical activity, maintaining healthy sleeping habits or a healthy weight, limiting alcohol or caffeine intake, or quitting smoking.

Oral strategies 620 action strategies are recommended to users 150, if the sleep apnea predicted by the model for the subject, is moderate apnea. These can include recommending oral devices that can be placed in the mouth to prevent blocked airways of the subjects while they are asleep. These can include dental appliances or oral mandibular advancement devices that prevent the tongue from blocking the throat and/or advancing the lower jaw forward. In addition, the healthcare provider can also recommend oral medications to the subjects to treat moderate sleep apnea which includes medications such as Acetazolamide, medroxyprogesterone, fluoxetine and protriptyline etc. The health care provider can also recommend exercises for mouth and facial muscles, called orofacial therapy, which may also be an effective treatment for sleep apnea in children and adults.

Breathing device 625 action strategies are recommended to users 150, if the sleep apnea predicted by the model for the subject, is severe apnea. To treat severe sleep apnea, the healthcare provider can recommend breathing devices which can include Continuous Positive Airway Pressure (CPAP) machine, an auto-adjusting Positive Airway Pressure (APAP) machine or a Bilevel Positive Airway Pressure (BPAP) machine. These machines deliver air pressure through a mask while sleeping. With CPAP, the air pressure is greater than that of the surrounding air and is just enough to keep the subject's upper airway passages open, preventing apnea and snoring. APAP is a type of airway pressure device that automatically adjusts the pressure while sleeping. BPAP machine supplies bilevel positive airway pressure and provides more pressure when subjects inhale and less when they exhale. The healthcare provider can recommend a particular breathing device based on the subject's needs, preferences, and face shapes to recommend the one that will work for the subject to treat the sleep apnea.

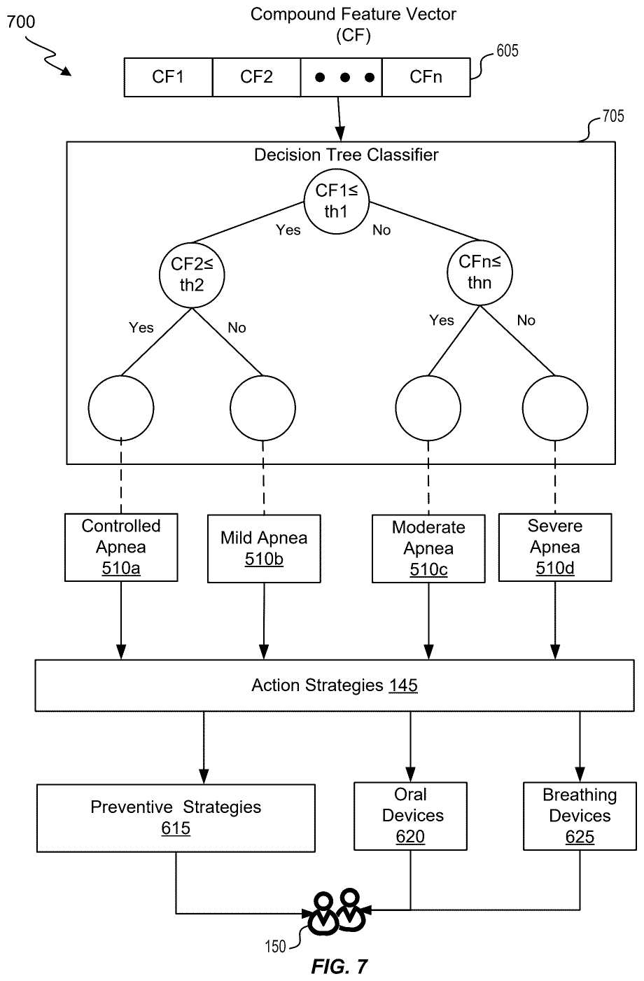

FIG. 7 illustrates another example process flow of the present disclosure, where a decision tree classifier can be used to generate sleep apnea classification categories using the compound features vector and generating respective action strategies. The example embodiment may be implemented by system 700 that includes a Compound Feature vector (CF) 605, a decision tree classifier 705, classification categories such as controlled apnea 510a, mild apnca 510b, moderate apnea 510c, severe apnea 510d, action strategies 145, preventive strategies 615, oral strategies 620, breathing device 625, and users 150.

Decision tree classifier 705 uses compound feature vector to train a decision tree machine-learning model to generate sleep apnea classification categories. A decision tree is a non-parametric supervised learning model used for classification and regression tasks. It has a hierarchical tree structure including a root node, branches, internal nodes, and leaf nodes. A decision tree model is used in decision support that depicts decisions and their potential outcomes. The decision tree model identifies the most significant compound feature among the set of compound features in the compound feature vector that contributes to the final decision and selects that as a root node of the tree. The dataset is then partitioned into subsets based on the compound feature values of the root node. Decision nodes are the resulting nodes from the splitting of root node and represent intermediate decisions or conditions within the tree. For example, compound feature 1, herein referred to as CF1, can be selected as a root node and based on the comparison with a threshold, herein referred to as th1. If the condition (CF1≤th1) is true, then it goes to the next node attached to that decision (Yes or No branch). The model picks the next significant compound feature to evaluate at the next level. The process is repeated until a stopping criterion is met and leaf nodes are generated which indicates the final classification category. Various statistical metrics can be employed to determine which compound feature is picked as the root node and the at the subsequent decision nodes. Examples of statistical metrics include Entropy (amount of uncertainty in the dataset), Information Gain (decrease in entropy), Gini Index (measure of degree of a variable being wrongly classified when it is randomly chosen), Gain ratio (measure that considers both the information gain and the number of outcomes a feature to determine the best feature among the set of features to split on) etc. For predicting a classification category for a record of the subject, the model starts from the root of tree, compares the compound feature values of the root node with compound feature value of another subject. Based on the result of a comparison, corresponding branches are followed till a leaf node is reached which represents the classification category.

The decision tree leaf nodes represent classification categories of sleep apnea which can include controlled apnea 510a, mild apnea 510b, moderate apnea 510c, and severe apnea 510d. Based on the predicted classification category action strategies 145 can be recommended to user 150. Action strategies 145 can be preventive strategies 615 for set of subjects predicted with controlled apnea 510a or mild apnea 510b, oral strategies 620 for set of subjects predicted with moderate apnea 510c, and/or breathing devices 625 for set of subjects predicted with severe apnea 510d. This results in an efficient and accurate decision system for the management of sleep apnea by empowering the set of subjects to make educated decisions about their health and well-being and to take precautionary measures before time.

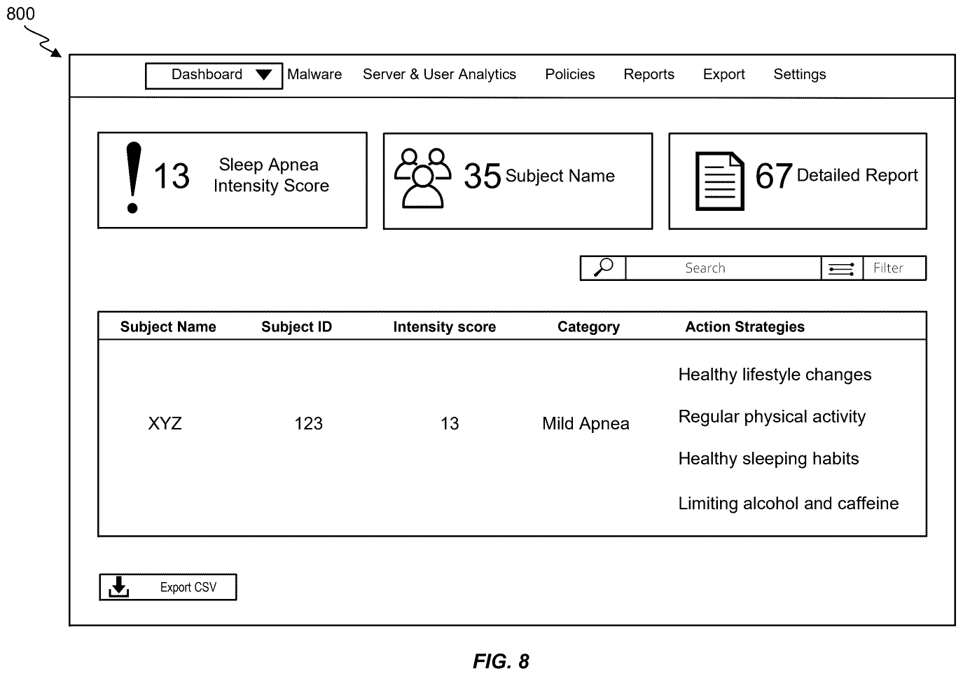

FIG. 8 displays a Graphical User Interface (GUI) that is displayed to the subject to get insights into sleep apnea conditions and to learn about the intensity of it so that respective actions can be taken to deal with the problems related to the sleep apnea. A dashboard 800 may display the detailed record of each subject after the analysis of the data has been effectively performed by using machine-learning models.

The dashboard may descriptively hold various sections where the subject's information may be displayed elaboratively. The sleep apnea intensity score block displays the intensity score of the respective subject along with the subject's name and detailed report. As the name indicates, the detailed report section shows the descriptive report of each subject of a set of subjects including the previous EMR and the updated health records. The panel also shows a table showing the records of the subject. The table may include a column like Subject name, Subject ID, Intensity Score, Category and Action Strategies. Subject name displays the name of the subject, Subject ID displays the unique ID that each subject gets in the initial phase of analysis to maintain confidentiality and the integrity of each subject's data. The Intensity Score shows the sleep apnea intensity score of the subject. As this score helps to differentiate and categorize the subject into different types of sleep apnea they may suffer from. As each category of a set of categories of sleep apnea has a different effect on a subject, each one may be dealt with unique actions and strategy plans. In the present disclosure, it has been described that the sleep apnea intensity score generated by the machine-learning model can be mapped to a category of sleep apnea such as controlled apnea, mild apnea, moderate apnea, and severe apnea. In addition to the intensity score, the category in which the subject lies are also displayed on the dashboard along with the actions recommended to treat that type of sleep apnea.

The advantage of this dashboard is that it displays the detailed insight of the subject's condition, which clearly informs the subject about the severity of sleep apnea and may also aid the doctors and the health care workers to have a better understanding of the apnea situation of a subject, which may help in treating the subject in a reliable, timely and accurate manner.

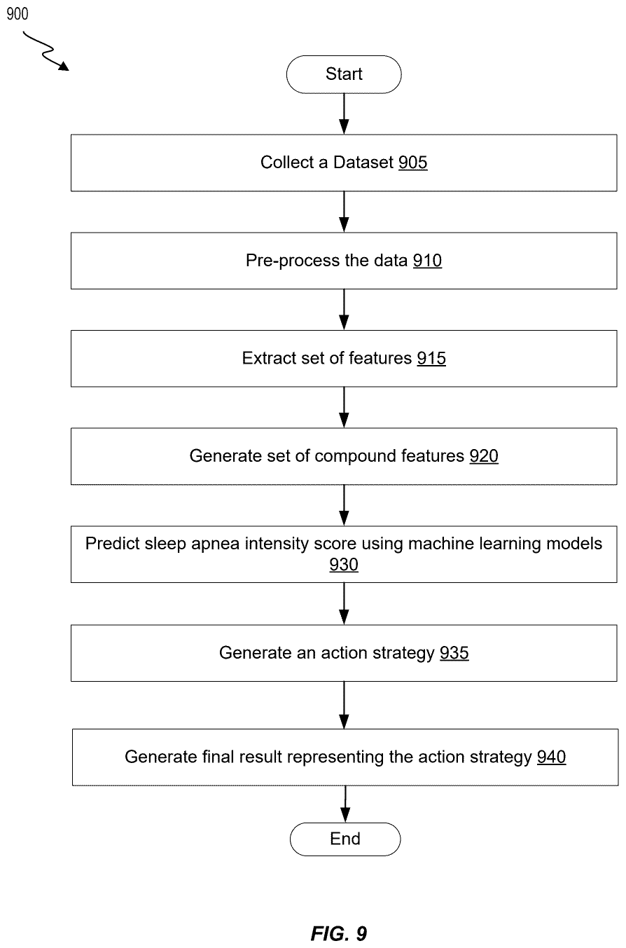

FIG. 9 illustrates an example process flow of a computer-implemented method 900 according to an example embodiment. Referring to FIG. 9, at block 905, a dataset is collected from a variety of data sources such as health data (electronic medical records), biometric sensors data, or audio data. Dataset 905 may include text data, time series data or audio data. At block 910, the collected dataset is preprocessed to enhance the quality of the data and ensure data consistency. It includes a range of operations, including noise reduction, data normalization, and data cleaning. At block 915, feature extraction is performed to extract a relevant set of features from the dataset. These features are qualities or traits found in the data that can be suggestive of sleep apnea. By concentrating on the relevant features, feature extraction streamlines the dataset and improves its analytical readability. At block 920, compound features are generated by performing arithmetic operations on two or more features. Compound features can enhance the predictive capabilities of the model by capturing complex relationships within the dataset. At block 930, ML model 135 is used to predict the sleep apnea intensity score by identifying various patterns and studying the compound features. The model analyzes the preprocessed data and the extracted features to generate an intensity score. The intensity score quantifies the severity of sleep apnea and can guide the decision-making process in an informed manner. Machine-learning models can include Random Forests, Support Vector Machine, AdaBoost, or Regression models etc. At block 935, action strategies 145 are generated based on the prediction of the intensity score estimated by the machine-learning model. At block 940, the result is generated in the form of an action strategy. The result may include converting recommendations and interventions into doable actions for the subject to treat the subject's sleep apnea condition efficiently and effectively.

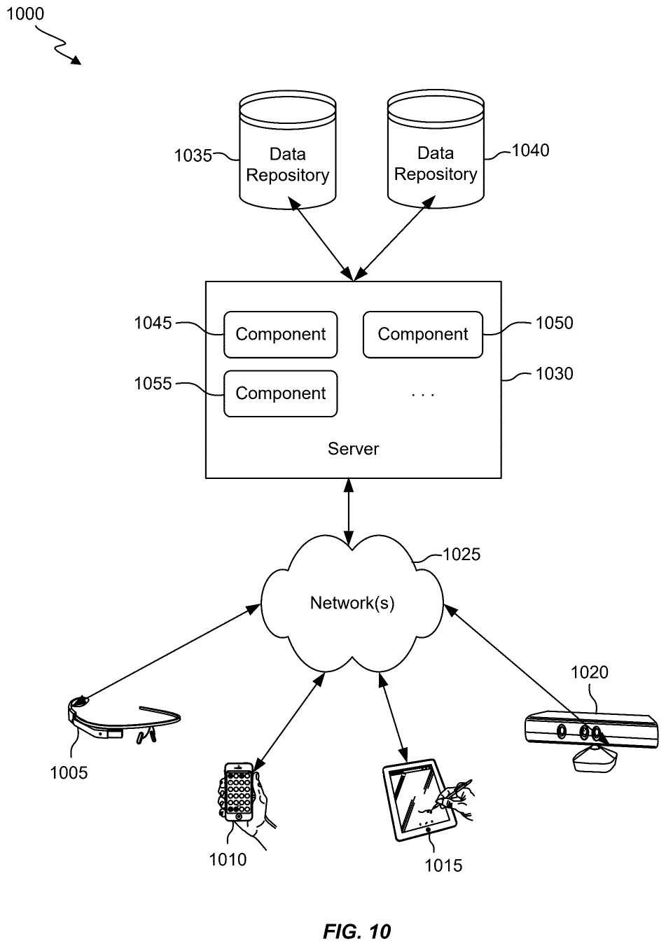

FIG. 10 depicts a simplified diagram of a distributed system 1000 for implementing system 100 of FIG. 1. In the illustrated embodiment, distributed system 1000 includes one or more subject computing devices 1005, 1010, 1015, and 1020, coupled to a server 1030 via one or more network(s) 1025. Subjects computing devices 1005, 1010, 1015, and 1020 may be configured to execute one or more applications.

In various aspects, server 1030 may be adapted to run one or services or software applications that enable techniques for determining sleep apnea from a dataset gathered from different data sources. In certain aspects, server 1030 may also provide other services or software applications that can include non-virtual and virtual environments. In some respects, these services may be offered as web-based or cloud services, such as under a Software as a Service (SaaS) model to the users of subject computing devices 1005, 1010, 1015, and/or 1020. Users operating subject computing devices 1005, 1010, 1015, and/or 1020 may, in turn, utilize one or more subject applications to interact with server 1030 to utilize the services provided by these components. Furthermore, subject computing devices 1005, 1010, 1015, and/or 1020 may, in turn, utilize one or more subject applications for prediction of sleep apnea.

In the configuration depicted in FIG. 10, server 1030 may include one or more components 1045, 1050, and 1055 that implement the functions performed by server 1030. These components may include software components that may be executed by one or more processors, hardware components, or combinations thereof. It may be appreciated that various system configurations are possible, which may be different from distributed system 1000. The embodiment shown in FIG. 10 is thus one example of a distributed system for implementing an embodiment system and is not intended to be limiting.

Users may use subject computing devices 1005, 1010, 1015, and/or 1020 for determining sleep apnea prediction from the dataset collected from a variety of data sources using various machine-learning models such as Random Forest model, Support Vector Machine model, AdaBoost model, etc. in accordance with the teachings of this disclosure. A subject device may provide an interface that enables a user of the subject device to interact with the subject device. The subject device may also output information to the user via this interface. Although FIG. 10 depicts only four subject computing devices, any number of subject computing devices may be supported.

The subject devices may include various types of computing systems such as portable handheld devices, general purpose computers such as personal computers and laptops, workstation computers, wearable devices, gaming systems, thin subjects, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone®), tablets (e.g., iPad®), personal digital assistants (PDAs), and the like. Wearable devices may include Google Glass® head-mounted displays and other devices. Gaming systems may include various handheld gaming devices, Internet-enabled gaming devices (e.g., a Microsoft Xbox® gaming console with or without a Kinect® gesture input device, Sony PlayStation® system, various gaming systems provided by Nintendo®, and others), and the like. The subject devices may be capable of executing various applications such as various Internet-related apps and communication applications (e.g., E-mail applications, short message service (SMS) applications) and may use various communication protocols.

Network(s) 1025 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) 1025 can be a Local Area Network (LAN), network based on Ethernet, Token-Ring, a Wide-Area Network (WAN), the Internet, a virtual network, a Virtual Private Network (VPN), an intranet, an extranet, a Public Switched Telephone Network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 1030 may include one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 1030 can include one or more virtual machines running virtual operating systems, or other computing architectures involving virtualization such as one or more flexible pools of logical storage devices that can be virtualized to maintain virtual storage devices for the server. In various aspects, server 1030 may be adapted to run one or more services or software applications that provide the functionality described in the foregoing disclosure.

The computing systems in server 1030 may run one or more operating systems including any of those discussed above, as well as any commercially available server operating system. Server 1030 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation, those commercially available from Oracle®, Microsoft®, Sybase®, IBM® (International Business Machines), and the like.

In some implementations, server 1030 may include one or more applications to implement various machine-learning algorithms. The data in database 105 of FIG. 1 may include data of various forms such as text data, audio data, time-series data, and real-time data. As an example, in a case where the data samples are text or image that may include but are not limited to, Twitter® feeds, Facebook® updates, or real-time updates received from one or more third-party information sources and continuous data streams, which may include real-time events related to sensor data applications, financial tickers, network performance measuring tools (e.g., network monitoring and traffic management applications), clickstream analysis tools, automobile traffic monitoring, and the like. Server 1030 may also include one or more applications to display the output of various processes of system 100 via one or more display devices of subject computing devices 1005, 1010, 1015, and 1020.

Distributed system 1000 may also include one or more data repositories 1035, and 1040. These data repositories may be used to store data in database 105 and other information in certain aspects. Data repositories 1035, and 1040 may reside in a variety of locations. For example, a data repository used by server 1030 may be local to server 1030 or may be remote from server 1030 and in communication with server 1030 via a network-based or dedicated connection. Data repositories 1035, and 1040 may be of different types. In certain aspects, a data repository used by server 1030 may be a database, for example, a relational database, such as databases provided by Oracle Corporation® and other vendors. One or more of these databases may be adapted to enable storage, update, and retrieval of data to and from the database in response to Structured Query Language (SQL)-formatted commands.

In certain aspects, one or more data repositories 1035, and 1040 may also be used by applications to store application data. The data repositories used by applications may be of different types such as, for example, a key-value store repository, an object store repository, or a general storage repository supported by a file system.

In certain aspects, the techniques for determining sleep apnea prediction from the dataset collected from a variety of data sources using various machine-learning models described in this disclosure may be offered as services via a cloud environment. FIG. 10 is a simplified block diagram of a cloud-based system environment in which various services of server 1030 of FIG. 10 may be offered as cloud services, in accordance with certain aspects. In the embodiment depicted in FIG. 10, subject computing devices 1005 may provide one or more cloud services that may be requested by users using one or more subject computing devices 1010, 1015, and 1020. Subject computing devices 1005 may comprise one or more computers and/or servers that may include those described for server 1030. The computers in subject computing devices 1005 may be organized as general-purpose computers, specialized server computers, server farms, server clusters, or any other appropriate arrangement and/or combination.

Network(s) 1025 may facilitate communication and exchange of data between subject computing devices 1010, 1015, and 1020 and subject computing devices 1005. Network(s) 1025 may include one or more networks. The networks may be of the same or different types. Network(s) 1025 may support one or more communication protocols, including wired and/or wireless protocols, for facilitating communications.

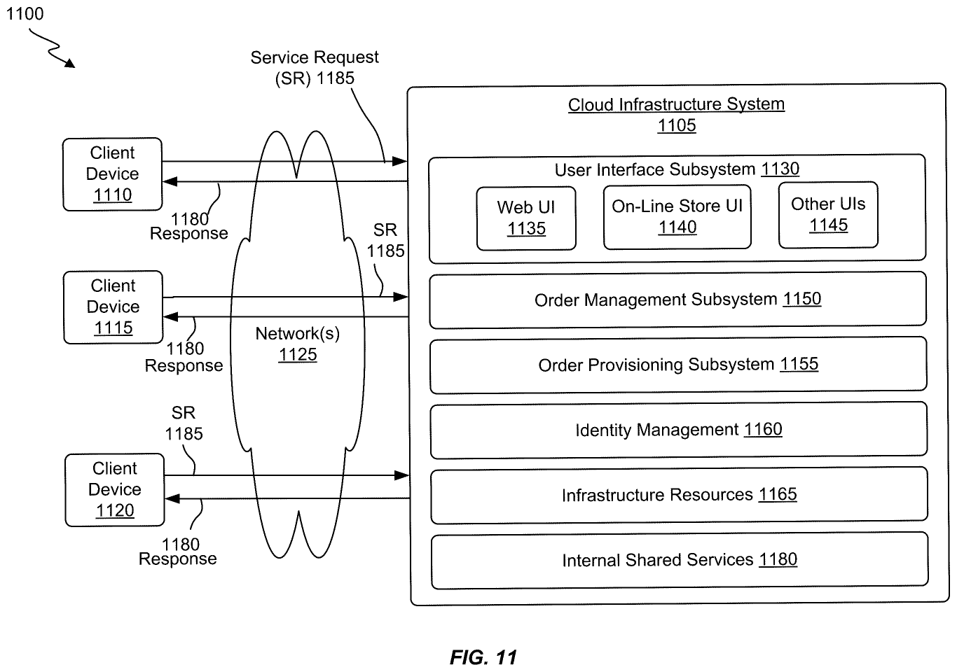

The embodiment depicted in FIG. 11 is only one example of a cloud infrastructure system and is not intended to be limiting. It should be appreciated that, in some other respects, cloud infrastructure system 1105 may have more or fewer components than those depicted in FIG. 11, may combine two or more components, or may have a different configuration or arrangement of components. For example, although FIG. 11 depicts three subject computing devices, any number of subject computing devices may be supported in alternative aspects.

The term cloud service is generally used to refer to a service that is made available to users on demand and via a communication network such as the Internet by systems (e.g., cloud infrastructure system 1105) of a service provider. Typically, in a public cloud environment, servers and systems that make up the cloud service provider's system are different from the subject's own on-premises servers and systems. The cloud service provider's systems are managed by the cloud service provider. Subjects can thus avail themselves of cloud services provided by a cloud service provider without having to purchase separate licenses, support, or hardware and software resources for the services. For example, a cloud service provider's system may host an application, and a user may, via a network 1125 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation® of Redwood Shores, California, such as middleware services, database services, Java cloud services, and others.

In certain aspects, cloud infrastructure system 1105 may provide one or more cloud services using different models such as under a Software as a Service (SaaS) model, a Platform as a Service (PaaS) model, an Infrastructure as a Service (IaaS) model, and others, including hybrid service models. Cloud infrastructure system 1105 may include a suite of applications, middleware, databases, and other resources that enable the provision of the various cloud services.

A SaaS model enables an application or software to be delivered to a subject over a communication network like the Internet, as a service, without the subject having to buy the hardware or software for the underlying application. For example, a SaaS model may be used to provide subjects access to on-demand applications that are hosted by cloud infrastructure system 1105. Examples of SaaS services provided by Oracle Corporation® include, without limitation, various services for human resources/capital management, subject relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a subject as a cloud service to provide elastic compute and storage capabilities. Various IaaS services are provided by Oracle Corporation®.

A PaaS model is generally used to provide, as a service, platform, and environment resources that enable subjects to develop, run, and manage applications and services without the subject having to procure, build, or maintain such resources. Examples of PaaS services provided by Oracle Corporation® include, without limitation, Oracle Java Cloud Service (JCS), Oracle Database Cloud Service (DBCS), data management cloud service, various application development solutions services, and others.

Cloud services are generally provided on an on-demand self-service basis, subscription-based, elastically scalable, reliable, highly available, and secure manner. For example, a subject, via a subscription order, may order one or more services provided by cloud infrastructure system 1105. Cloud infrastructure system 1105 then performs processing to provide the services requested in the subject's subscription order. Cloud infrastructure system 1105 may be configured to provide one or even multiple cloud services.

Cloud infrastructure system 1105 may provide cloud services via different deployment models. In a public cloud model, cloud infrastructure system 1105 may be owned by a third-party cloud services provider and the cloud services are offered to any general public subject, where the subject can be an individual or an enterprise. In certain other aspects, under a private cloud model, cloud infrastructure system 1105 may be operated within an organization (e.g., within an enterprise organization) and services provided to subjects that are within the organization. For example, the subjects may be various departments of an enterprise, such as the Human Resources department, the payroll department, etc. or even individuals within the enterprise. In certain other aspects, under a community cloud model, the cloud infrastructure system 1105 and the services provided may be shared by several organizations in a related community. Various other models, such as hybrids of the above-mentioned models may also be used.

Subject computing devices 1110, 1115, and 1120 may be of several types (such as cloud infrastructure system 1105, 1110, 1115, and 1120 depicted in FIG. 11) and may be capable of operating one or more subject applications. A user may use a subject device to interact with Cloud Infrastructure System 1105, such as to request a service provided by Cloud Infrastructure System 1105.

As depicted in the embodiment in FIG. 11, cloud infrastructure system 1105 may include infrastructure resources 1175 that can be utilized for facilitating the provision of various cloud services offered by cloud infrastructure system 1105. These services include 910, 915, 920, 925, 930, 935, and 940 as shown in FIG. 9. Infrastructure resources 1175 may include, for example, processing resources, storage or memory resources, networking resources, and the like.

In certain aspects, to facilitate efficient provisioning of these resources for supporting the various cloud services provided by cloud infrastructure system 1105 for different subjects, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1105 may itself internally use services 1170 that are shared by different components of cloud infrastructure system 1105 and which facilitate the provisioning of services by cloud infrastructure system 1105. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 1105 may comprise multiple subsystems. These subsystems may be implemented in software, or hardware, or combinations thereof. As depicted in FIG. 11, the subsystems may include a user interface subsystem 1130 that enables users or subjects of cloud infrastructure system 1105 to interact with cloud infrastructure system 1105. User interface subsystem 1130 may include various interfaces such as a web interface 1135, an online store interface 1140 where cloud services provided by cloud infrastructure system 1105 are advertised and are purchasable by a consumer, and other interfaces 1145. For example, a subject may, using a subject device, request (service request 1175) one or more services provided by cloud infrastructure system 1105 using one or more of interfaces 1135, 1140, and 1145. For example, a subject may access the online store, browse cloud services offered by cloud infrastructure system 1105, and place a subscription order for one or more services offered by cloud infrastructure system 1105 that the subject wishes to subscribe to. The service request may include information identifying the subject and one or more services that the subject desires to subscribe to. For example, a subject may place a subscription order for a Chabot related service offered by cloud infrastructure system 1105. As part of the order, the subject may provide information identifying for input (e.g., utterances).

In certain aspects, such as the embodiment depicted in FIG. 11, cloud infrastructure system 1105 may comprise an Order Management Subsystem (OMS) 1150 that is configured to process the new order. As part of this processing, OMS 1150 may be configured to: create an account for the subject, if not done already; receive billing and/or accounting information from the subject that is to be used for billing the subject for providing the requested service to the subject; verify the subject information; upon verification, book the order for the subject; and orchestrate various workflows to prepare the order for provisioning.

Once properly validated, OMS 1150 may then invoke Order Provisioning Subsystem (OPS) 1155 that is configured to provision resources for the order including processing, memory, and networking resources. The provisioning may include allocating resources for the order and configuring the resources to facilitate the service requested by the subject order. The manner in which resources are provisioned for an order and the type of the provisioned resources may depend upon the type of cloud service that has been ordered by the subject. For example, according to one workflow, OPS 1155 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting subject for providing the requested service.

Cloud infrastructure system 1105 may send a response or notification 1190 to the requesting subject to indicate when the requested service is now ready for use. In some instances, information (e.g., a link) may be sent to the subject that enables the subject to start using and availing the benefits of the requested services.

Cloud infrastructure system 1105 may provide services to multiple subjects. For each subject, cloud infrastructure system 1105 is responsible for managing information related to one or more subscription orders received from the subject, maintaining subject data related to the orders, and providing the requested services to the subject. Cloud infrastructure system 1105 may also collect usage statistics regarding a subject's use of subscribed services. For example, statistics may be collected for the amount of storage used, the amount of data transferred, the number of users, and the amount of system up time and system down time, and the like. This usage information may be used to bill the subject. Billing may be done, for example, on a monthly cycle.

Cloud infrastructure system 1105 may provide services to multiple subjects in parallel. Cloud infrastructure system 1105 may store information for these subjects, including possibly proprietary information. In certain aspects, cloud infrastructure system 1105 comprises an identity management subsystem Identity Management Subsystem (IMS) 1170 that is configured to manage the subject's information and provide the separation of the managed information such that information related to one subject is not accessible by another subject. IMS 1170 may be configured to provide various security-related services such as identity services, such as information access management, authentication and authorization services, services for managing subject identities and roles and related capabilities, and the like.

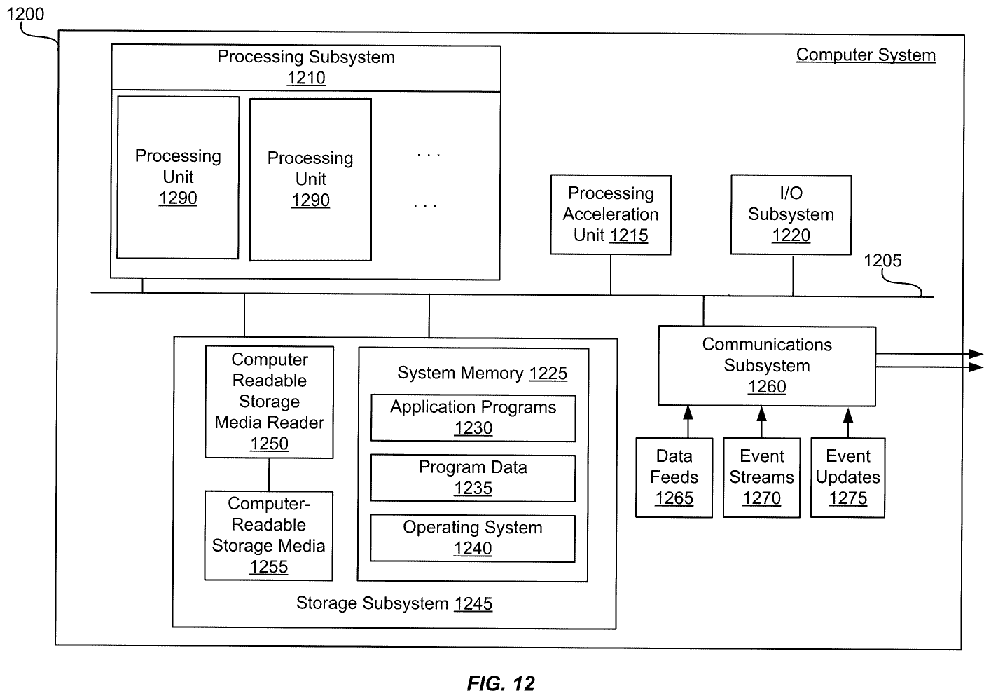

FIG. 12 illustrates an exemplary computer system 1200 that may be used to implement certain aspects of system 100 for sleep apnea detection. For example, in some respects, computer system 1200 may be used to implement any of the systems for determining sleep apnea prediction from the dataset collected from a variety of data sources using various machine-learning models shown in FIG. 1 and various servers and computer systems described above. As shown in FIG. 12, computer system 1200 includes various subsystems including a processing subsystem 1210 that communicates with a few other subsystems via a bus subsystem 1205. These other subsystems may include a processing acceleration unit 1215, and I/O subsystem 1220, a storage subsystem 1245, and a communications subsystem 1270. Storage subsystem 1245 may include non-transitory computer-readable storage media including storage media 1255 and a system memory 1225.

Bus subsystem 1205 provides a mechanism for letting the various components and subsystems of computer system 1200 communicate with each other as intended. Although bus subsystem 1205 is shown schematically as a single bus, alternative aspects of the bus subsystem may utilize multiple buses. Bus subsystem 1205 may be any of several types of bus structures including a memory bus or memory controller, a peripheral bus, a local bus using any of a variety of bus architectures, and the like. For example, such architectures may include an Industry Standard Architecture (ISA) bus, a Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P13127.1 standard, and the like.

Processing subsystem 1210 controls the operation of computer system 1200 and may comprise one or more processors, Application Specific Integrated Circuits (ASICs), or Field Programmable Gate Arrays (FPGAs). The processors may include single-core, or multicore processors. The processing resources of computer system 1200 can be organized into one or more processing units 1290, 1280, etc. A processing unit may include one or more processors, one or more cores from the same or different processors, a combination of cores and processors, or other combinations of cores and processors. In some embodiments, processing subsystem 1210 can include one or more special-purpose co-processors such as graphics processors, digital signal processors (DSPs), or the like. In some embodiments, some or all of the processing units of processing subsystem 1210 can be implemented using customized circuits, such as ASICs, or FPGAs.

In some embodiments, the processing units in processing subsystem 1210 can execute instructions stored in system memory 1225 or on computer-readable storage media 1255. In various aspects, the processing units can execute a variety of programs or code instructions and can maintain multiple concurrently executing programs or processes. At any given time, some, or all of the program code to be executed can be resident in system memory 1225 and/or on computer-readable storage media 1255 including potentially on one or more storage devices. Through suitable programming, processing subsystem 1210 can provide various functionalities described above. In instances where computer system 1200 is executing one or more virtual machines, one or more processing units may be allocated to each virtual machine.

In certain aspects, a processing acceleration unit 1215 may optionally be provided for performing customized processing or for off-loading some of the processing performed by processing subsystem 1210 to accelerate the overall processing performed by computer system 1200.

I/O subsystem 1220 may include devices and mechanisms for inputting information to computer system 1200 and/or for outputting information from or via computer system 1200. In general, use of the term input device is intended to include all possible types of devices and mechanisms for inputting information to computer system 1200. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Microsoft Kinect® motion sensor that enables users to control and interact with an input device, the Microsoft Xbox® 370 game controller, devices that provide an interface for receiving input using gestures and spoken commands. User interface input devices may also include eye gesture recognition devices such as the Google Glass® blink detector that detects eye activity (e.g., “blinking” while taking pictures and/or making a menu selection) from users and transforms the eye gestures as inputs to an input device (e.g., Google Glass®). Additionally, user interface input devices may include voice recognition sensing devices that enable users to interact with voice recognition systems (e.g., Siri® navigator) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 1200 to a user or other computer. User interface output devices may include a display subsystem, indicator lights, or non-visual displays such as audio output devices, etc. The display subsystem may be a Cathode Ray Tube (CRT), a flat-panel device, such as that using a Liquid Crystal Display (LCD) or plasma display, a projection device, a touch screen, and the like. For example, user interface output devices may include, without limitation, a variety of display devices that visually convey text, graphics, and audio/video information such as monitors, printers, speakers, headphones, automotive navigation systems, plotters, voice output devices, and modems.

Storage subsystem 1245 provides a repository or data store for storing information and data that is used by computer system 1200. Storage subsystem 1245 provides a tangible non-transitory computer-readable storage medium for storing the basic programming and data constructs that provide the functionality of some aspects. Storage subsystem 1245 may store software (e.g., programs, code modules, instructions) that, when executed by processing subsystem 1210 provides the functionality described above. The software may be executed by one or more processing units of processing subsystem 1210. Storage subsystem 1245 may also provide a repository for storing data used in accordance with the teachings of this disclosure.

Storage subsystem 1245 may include one or more non-transitory memory devices, including volatile and non-volatile memory devices. As shown in FIG. 12, storage subsystem 1245 includes a system memory 1225 and a computer-readable storage media 1255. System memory 1225 may include a number of memories including a volatile main random-access memory (RAM) for storage of instructions and data during program execution and a non-volatile Read Only Memory (ROM) or flash memory in which fixed instructions are stored. In some implementations, a basic input/output system (BIOS), containing the basic routines that help to transfer information between elements within computer system 1200, such as during start-up, may typically be stored in the ROM. The RAM typically contains data and/or program modules that are presently being operated and executed by processing subsystem 1210. In some implementations, system memory 1225 may include multiple different types of memory, such as Static Random Access Memory (SRAM), Dynamic Random Access Memory (DRAM), and the like.

By way of example, and not limitation, as depicted in FIG. 12, system memory 1225 may load application programs 1230 that are being executed, which may include various applications such as Web browsers, mid-tier applications, Relational Database Management Systems (RDBMS), etc., program data 1235, and an operating system 1240. By way of example, operating system 1240 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, Palm® OS operating systems, and others.

Computer-readable storage media 1255 may store programming and data constructs that provide the functionality of some aspects. Computer-readable media 1255 may provide storage of computer-readable instructions, data structures, program modules, and other data for computer system 1200. Software (programs, code modules, instructions) that, when executed by processing subsystem 1210 provides the functionality described above, may be stored in storage subsystem 1245. By way of example, computer-readable storage media 1255 may include non-volatile memory such as a hard disk drive, a magnetic disk drive, an optical disk drive such as a CD ROM, Digital Video Disc (DVD), a Blu-Ray® disk, or other optical media. Computer-readable storage media 1255 may include, but is not limited to, Zip® drives, flash memory cards, Universal Serial Bus (USB) flash drives, secure digital (SD) cards, DVD disks, digital video tape, and the like. Computer-readable storage media 1255 may also include, Solid-State Drives (SSD) based on non-volatile memory such as flash-memory based SSDs, enterprise flash drives, solid state ROM, and the like, SSDs based on volatile memory such as solid state RAM, dynamic RAM, static RAM, Dynamic Random Access Memory (DRAM)-based SSDs, magneto resistive RAM (MRAM) SSDs, and hybrid SSDs that use a combination of DRAM and flash memory based SSDs.

In certain aspects, storage subsystem 1245 may also include a computer-readable storage media reader 1250 that can further be connected to computer-readable storage media 1255. Reader 1250 may receive and be configured to read data from a memory device such as a disk, a flash drive, etc.

In certain aspects, Computer System 1200 may support virtualization technologies, including but not limited to the virtualization of processing and memory resources. For example, computer system 1200 may provide support for executing one or more virtual machines. In certain aspects, Computer System 1200 may execute a program such as a hypervisor that facilitates the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1200. Accordingly, multiple operating systems may potentially be run concurrently by Computer System 1200.

Communications subsystem 1270 provides an interface to other computer systems and networks. Communications subsystem 1270 serves as an interface for receiving data from and transmitting data to other systems from computer system 1200. For example, communications subsystem 1270 may enable computer system 1200 to establish a communication channel to one or more subject devices via the Internet for receiving and sending information from and to the subject devices. For example, the communication subsystem may be used to transmit a response to a user regarding the inquiry for a Chabot.

Communication subsystem 1270 may support both wired and/or wireless communication protocols. For example, in certain aspects, communications subsystem 1270 may include Radio Frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 1202.XX family standards, or other mobile communication technologies, or any combination thereof), Global Positioning System (GPS) receiver components, and/or other components. In some aspects communications subsystem 1270 can provide wired network connectivity (e.g., Ethernet) in addition to or instead of a wireless interface.

Communication subsystem 1270 can receive and transmit data in various forms. For example, in some embodiments, in addition to other forms, communications subsystem 1270 may receive input communications in the form of structured and/or unstructured data feeds 1275, event streams 1270, event updates 1275, and the like. For example, communications subsystem 1270 may be configured to receive (or send) data feeds 1275 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 1270 may be configured to receive data in the form of continuous data streams, which may include event streams 1270 of real-time events and/or event updates 1275, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1270 may also be configured to communicate data from computer system 1200 to other computer systems or networks. The data may be communicated in various forms such as structured and/or unstructured data feeds 1275, event streams 1270, event updates 1275, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a personal digital assistant (PDA)), a wearable device (e.g., a Google Glass® head mounted display), a personal computer, a workstation, a mainframe, a kiosk, a server rack, or any other data processing system. Due to the ever-changing nature of computers and networks, the description of computer system 1200 depicted in FIG. 12 is intended only as a specific example. Many other configurations having more or fewer components than the system depicted in FIG. 12 are possible. Based on the disclosure and teachings provided herein, a person of ordinary skill in art can appreciate other ways and/or methods to implement the various aspects.

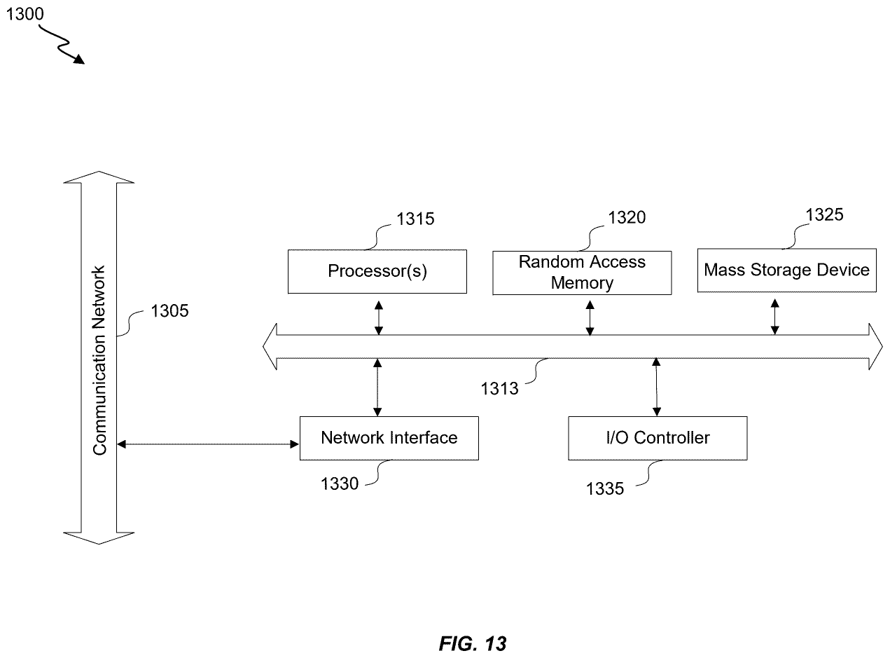

FIG. 13 illustrates an exemplary server which may include a processor, a memory, and a mass storage device. An exemplary Server 1300 may include a processor 1315, a memory (e.g., RAM) 1320, a bus 1310 which couples processor 1315 and memory 1320, a mass storage device 1325 (e.g., a magnetic or optical disk) coupled to processor 1315 and memory 1320 through an I/O controller 1335, and a network interface 1330 coupled to the processor and the memory. Network interface 1330 is further connected to a communication network 1305. Servers may be clustered together to handle more subject traffic and may include separate servers for different functions such as a database server, an application server, and a Web presentation server. Such servers may further include one or more mass storage devices 1325 such as a disk farm or a redundant array of independent disk (“RAID”) system for additional storage and data integrity. Read-only devices, such as compact disk drives and digital versatile disk drives, may also be connected to the servers. Suitable servers and mass storage devices are manufactured by, for example, Compaq, IBM, and Sun Microsystems. Generally, a server may operate as a source of content and provide any associated back-end processing, while an end user can be consumer of content provided by the server. However, it should be appreciated that many of the devices described above may be configured to respond to remote requests, thus operating as a server, and the devices described as servers may operate as end users of remote data sources. In contemporary peer-to-peer networks and environments such as RSS environments, the distinction between end users and servers is a blur. Accordingly, as used herein, the term “server” as used herein is generally intended to refer to any of the above-described servers, or any other device that may be used to provide content such as RSS feeds in a networked environment.

Example 1: Purpose of this example is to showcase one of the example embodiments that includes evaluating feature importance of the features during the feature extraction 410b. The features include the set of features extracted from different data sources and generated compound features using two or more features. The example also showcases the example embodiment that includes training the machine-learning models using the identified important features and evaluates performance of the trained models. In one of the embodiments, a feature importance was run on the machine-learning model for example, the Random Forest model to check the important features and the top five important features are listed in Table 1. The table lists the feature name, description of the feature and weightage which quantifies the importance of a feature. As the results show, the compound feature ‘med_oxy_weight’ has the highest weightage of feature importance among the set of compound features with the value 0.2876. The relevant features identified during the feature importance step were used as input variables for training the Random Forest model.

TABLE 1
Feature importance of different features

| S No. | Feature name | Description | Weightage |
|---|---|---|---|
| 1 | med_oxy_weight | Measured by multiplying ratio of | 0.2876 |
| | | median oxygen levels by weight | |
| | | groups | |
| 2 | age_bmi | Individuals BMI value is | 0.1268 |
| | | multiplied by age groups | |
| 3 | hem_weight_med | Measuring O₂ proportion in | 0.0795 |
| | | hemoglobin and multiplying by | |
| | | weight class of patient (basically | |
| | | 4 classes of weight) across | |
| | | median O₂ level | |
| 4 | oxy_age_bmi | Measured by multiplying | 0.0327 |
| | | average O₂ levels of individual | |
| | | with age group divided by BMI | |
| 5 | Mean_sleep | The average number of hours a | 0.0234 |
| | | patient gets sleep each day | |

In one of the embodiments, several machine-learning models were implemented to predict sleep apnea in the subject. Various machine-learning models such as Random Forest model, AdaBoost model, Decision tree model, and Support Vector machine model were trained using the relevant features identified during feature importance as training dataset. 80 percent of the data was used for training, and 20 percent was used as test data. The implemented machine-learning model's performance results are shown in Table 2. Model performance is evaluated based on different parameters such as accuracy, Root Mean Squared Error (RMSE) and Mean Absolute Percentage Error (MAPE). Table 2 lists the implemented model name, and performance metrics such as accuracy, RMSE, and MAPE for each model. The results show that all the listed models accurately predict sleep apnea in the set of subjects with the model accuracy greater than 94%. The Random Forest model has the highest accuracy value and the lowest RMSE and MAPE values as compared to accuracy, RMSE, and MAPE values of AdaBoost, Decision tree, and Support Vector Machine model. This shows that Random Forest model outperforms all the other implemented machine-learning models in terms of the performance indicators such as accuracy, RMSE, and MAPE.

TABLE 2
Model performance results

| S No. | Model name | Accuracy | RMSE | MAPE |
|---|---|---|---|---|
| 1 | Random Forest | 0.9678 | <0.3 | <0.05 |
| 2 | AdaBoost | 0.9558 | <0.7 | <0.12 |
| 3 | Decision tree | 0.9455 | <0.9 | <0.13 |
| 4 | Support Vector Machine | 0.9347 | <0.12 | <0.15 |

Some embodiments of the present disclosure include a system including one or more data processors. In some embodiments, the system includes a non-transitory computer readable storage medium containing instruction which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, although the present invention as claimed has been specifically disclosed by embodiments and optional features, modification and variation of the concepts herein disclosed may be resorted to by those skilled in the art, and that such modifications and variations are within the scope of this invention as defined by the appended claims.

The present description provides preferred exemplary embodiments only, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

What is claimed:

1. A computer-implemented method comprising:
collecting a dataset from one or more data sources for a subject;
extracting a set of features from the dataset;
generating a compound feature from two or more features of the set of features;
predicting, for the subject, an intensity score for a sleep apnea by processing an input dataset that includes the compound feature using a machine-learning model;
generating an action strategy based on the intensity score of the sleep apnea predicted by the machine-learning model; and
outputting a result that represents the action strategy.

2. The computer-implemented method of claim 1, wherein the dataset includes data from an electronic health record.

3. The computer-implemented method of claim 1, wherein the dataset includes data from a sensor in a wearable device.

4. The computer-implemented method of claim 1, further comprising:
extracting from the dataset the set of features comprising of one or more demographic features, one or more comorbidities features, one or more anthropometric features or one or more sleep history features.

5. The computer-implemented method of claim 1, further comprising:
generating a second compound feature from two or more features of the set of features by performing arithmetic operations on the two or more features of the set of features.

6. The computer-implemented method of claim 5, wherein generating the second compound feature includes multiplying an O₂ proportion in hemoglobin feature and a weight class feature of the subject.

7. The computer-implemented method of claim 1, further comprising:
preprocessing the dataset, wherein the preprocessing comprises handling one or more missing values or converting a categorical set of features to a numerical set of features.

8. The computer-implemented method of claim 1, further comprising:
predicting the sleep apnea using the machine-learning model trained using training dataset that includes the compound feature associated with each subject of a set of subjects wherein the machine-learning model includes a Random Forest model, a Support Vector Machine model, or an AdaBoost model.

9. The computer-implemented method of claim 1, further comprising:
mapping the intensity score of the sleep apnea to a category of a set of categories of the sleep apnea, wherein the set of categories includes a controlled apnea, a mild apnea, a moderate apnea and a severe apnea.

10. The computer-implemented method of claim 9, wherein the category of the set of categories is the mild apnea, and wherein the computer-implemented method further comprises:
generating the action strategy of one or more preventive recommendation strategies for the subject.

11. The computer-implemented method of claim 9, wherein the category of the set of categories is the moderate apnea, and wherein the computer-implemented method further comprises:
generating the action strategy of oral or breathing devices recommendation strategies for the subject.

12. A system comprising:
one or more data processors; and
a non-transitory computer readable storage medium containing instructions which, when executed on the one or more data processors, cause the one or more data processors to perform actions including:
collecting a dataset from one or more data sources for a subject;
extracting a set of features from the dataset;
generating a compound feature from two or more features of the set of features;
predicting, for the subject, an intensity score for a sleep apnea by processing an input dataset that includes the compound feature using a machine-learning model;
generating an action strategy based on the intensity score of the sleep apnea predicted by the machine-learning model; and
outputting a result that represents the action strategy.

13. A computer-program product tangibly embodied in a non-transitory machine-readable storage medium, including instructions configured to cause one or more data processors to perform actions including:
collecting a dataset from one or more data sources for a subject;
extracting a set of features from the dataset;
generating a compound feature from two or more features of the set of features;
predicting, for the subject, an intensity score for a sleep apnea by processing an input dataset that includes the compound feature using a machine-learning model;
generating an action strategy based on the intensity score of the sleep apnea predicted by the machine-learning model; and
outputting a result that represents the action strategy.

14. The computer-program product of claim 13, wherein the actions further include:
extracting from the dataset the set of features comprising of one or more demographic features, one or more comorbidities features, one or more anthropometric features or one or more sleep history features.

15. The computer-program product of claim 13, wherein the actions further include:
generating a second compound feature from two or more features of the set of features by performing arithmetic operations on the two or more features of the set of features.

16. The computer-program product of claim 15, wherein generating the second compound feature includes multiplying an O₂ proportion in hemoglobin feature and a weight class feature of the subject.

17. The computer-program product of claim 13 wherein the actions further include:
predicting the sleep apnea using the machine-learning model trained using training dataset that includes the compound feature associated with each subject of a set of subjects wherein the machine-learning model includes a Random Forest model, a Support Vector Machine model, or an AdaBoost model.

18. The computer-program product of claim 13, wherein the actions further include:
mapping the intensity score of the sleep apnea to a category of a set of categories of the sleep apnea, wherein the set of categories includes a controlled apnea, a mild apnea, a moderate apnea and a severe apnea.

19. The computer-program product of claim 18, wherein the category of the set of categories is the mild apnea, and wherein the actions further include:
generating the action strategy of one or more preventive recommendation strategies for the subject.

20. The computer-program product of claim 13, wherein the actions further include:
preprocessing the dataset, wherein the preprocessing comprises handling one or more missing values or converting a categorical set of features to a numerical set of features.
